(19) United States
(12) Patent Application Publication
(10) Pub. No.: US 2005/0281786 A1
(43) Pub. Date: Dec. 22, 2005

(54) AAV mediated gene delivery to cochlear cells

(76) Inventors: David Poulsen, Missoula, MT (US); Peter VonDoersten, Missoula, MT (US); Diana Lurie, Missoula, MT (US); Ida Stone, Missoula, MT (US)

Correspondence Address: FENNEMORE CRAIG, 3003 NORTH CENTRAL AVENUE, SUITE 2600, PHOENIX, AZ, 85012, US

(21) Appl. No.: 11/155,964

(22) Filed: Jun. 17, 2005

Related U.S. Application Data
(60) Provisional application No. 60/580,752, filed on Jun. 18, 2004.

(51) Int. Cl. A61K 48/00; C12N 15/861

(52) U.S. Cl. 424/93.2; 435/456

(57) ABSTRACT

The present invention is directed to a method of transducing mammalian cochlear cells, more preferably, cochlear hair cells and support cells. The method involves the delivery of adeno-associated virus (AAV) to a target mammalian cochlear cell. The AAV comprises DNA which is exogenous to the AAV and a promoter operatively linked to the DNA. Preferably, the promoter is a cell specific promoter, e.g., hair cell or support cell specific promoter, and the AAV is serotype 1, 2, 6, or a mixture of two or more serotypes. The present invention also relates to compositions comprising modified AAV useful in transducing specific cochlear cells.

### CROSS-REFERENCE TO RELATED APPLICATIONS

This application claims the benefit of U.S. provisional application 60/580,752 filed Jun. 18, 2004, the content of which is expressly incorporated herein by reference thereto.

### STATEMENT REGARDING FEDERALLY SPONSORED RESEARCH OR DEVELOPMENT

This invention was supported in part by funds from the U.S. government (NIH R 21 DC05462 “Transduction of the mouse auditory system with AAV”). Therefore, the U.S. government may have certain rights in the invention.

### FIELD OF THE INVENTION

The present invention relates to methods of transducing mammalian cochlear cells using an adeno-associated virus (AAV). The present invention also relates to compositions comprising AAV for transducing cochlear hair cells and support cells.

### BACKGROUND OF THE INVENTION

More than 28 million Americans suffer from various forms of hearing loss and 30 million more are exposed to dangerous levels of noise. The lack of effective treatment for many forms of acquired and inherited hearing disorders has prompted interest in the potential application of newly developed gene delivery techniques to restore normal cochlear function. Gene transduction into the cochlea offers the potential for developing therapeutic strategies to treat both inherited and pathological hearing disorders. However, in order to develop a gene therapy strategy that will successfully treat hearing disorders, appropriate vectors must be developed that are capable of transducing cochlear hair cells and support cells, which are free of serious side effects.

Virus-mediated gene transfer into the cochlea has been previously accomplished with limited success. The previous methods failed to either transduce the hair cells and specific support cells or resulted in negative side effects, such as destruction of the transduced cells after treatment. For example, gene expression following transduction with lentivirus was restricted to cells lining the paralymphatic space (Han et al., 1999). Treatment with adenovirus in vivo resulted in the transduction of over 90% of inner hair cells, more than 50% of outer hair cells, and even some supporting pillar cells of the guinea pig (Stover et al., 2000; Luebke et al., 2001a, 2001b). Treatment with adenovirus, however, often results in the stimulation of an immune response that results in the ultimate destruction of the transduced cells and also often has a limited duration of transgene expression. These negative side effects are major drawbacks to using adenovirus for gene transfer.

AAV has several characteristics, which make it attractive as a gene delivery system (for review see Bueler, 1999, Carter and Samulski, 2000; During and Ashenden, 1998; Flotte et. al., 1996; Peel and Klein, 2000; Rabinowitz and Samulski, 2000; Snyder, 1999; Xiao et. al., 1997). AAV is a nonpathogenic human parvovirus that infects approximately 85% of humans within the first decade of life and has never been associated with disease. AAV also has an extremely broad host range, capable of infecting most cell types, including post-mitotic cells. Eight different AAV serotypes (AAV-1-8) have been identified based on amino acid sequence differences in their respective capsid proteins. Serotypes 1 and 6 share >99% amino acid homology and therefore are not functionally differentiated. Recombinant AAV has demonstrated transduction and long-term gene expression (up to 1.5 years, Carter and Samulski, 2000) in the liver, lung, muscle, brain, vasculature and retina of experimental animals (Rabinowitz and Samulski, 2000; Walters et al., 2001). Furthermore, AAV vectors have been used in a number of clinical trials with no apparent pathological effects on cell growth, morphology or differentiation. AAV serotype 2 was the first to be cloned and therefore has been used in the vast majority of gene transfer studies to date and the only serotype examined in the auditory system.

Even though AAV appears to be a favorable choice, previous studies using AAV in the auditory system indicated that AAV is not suitable and, in fact, unable to transduce cochlear hair cells or support cells—Dieter's cells, Hensen's cells, pillar cells, inner phalangeal cells, border cells, or interdential cells (Jero et al., 2001; Kho et al., 2000; Luebke et al., 2001b). It was speculated that this is due to the lack of heparin sulfate on the surface of these cells (Luebke et al., 2001b, “one explanation for the lack of hair cell transduction with AAV may be the absence of heparin sulfate proteoglycans on hair cells”).

In contrast to the prior art, the inventors surprisingly discovered, and provide herein, a method of transducing cochlear hair cells and support cells with AAV.

### SUMMARY OF THE INVENTION

The invention relates to a method of transducing mammalian cochlear cells, preferably hair cells or support cells. The method comprises the step of delivering an adeno-associated virus (AAV) to a target hair cell or support cell. The AAV used to transduce the cochlear cell is modified and comprises DNA that is exogenous to the AAV and operatively linked to a promoter.

Preferably, a high-titer AAV having a titer of at least 10⁹ genomic particles per μl (gp/μl) is used and, more preferably of at least 10¹⁰ gp/μl or alternatively at least 10¹¹ gp/μl.

In one embodiment, the exogenous DNA in the AAV encodes a protein that promotes cochlear hair cell growth, cell differentiation, e.g., promotes support cell differentiation into hair cells, or corrects a genetic mutation. In a preferred embodiment, the DNA encodes a Math1, Hath1, SOX2, connexin 26, or a growth factor protein. Specific examples of preferred growth factor proteins include: Nerve Growth Factor (NGF), Glial-Derived Neurotrophic Factor (GDNF), and Fibroblast Growth Factor (FGF).

In a preferred embodiment, the DNA encodes an ER receptor fusion protein, such as, Math1/ER, Hath1/ER, or SOX2/ER protein. In this embodiment, the method further comprises the step of administering an activator compound, such as, tamoxifen.

Preferably the invention further comprises the use of a specific promoter. Preferred promoters include cochlear hair cell specific promoters and support cell specific promoters. Examples of support cell specific promoters include the following: the glial fibrillary acidic protein (GFAP) promoter, the excitatory amino acid transporter-1 (EAAT1) promoter, the GLAST promoter and the murine cytomegalovirus (mCMV) promoter. In addition, examples of hair cell specific promoters include: the human cytomegalovirus (CMV) promoter, the chicken β-actin/CMV hybrid (CAG) promoter, and the myosin VIIA promoter. In one embodiment, the promoter used is the CAG promoter.

Advantageously, the AAV may also comprise a woodchuck hepatitis virus post-transcription regulatory element (WPRE).

In a preferred embodiment, the transduction efficiency of the disclosed method is at least 30%; preferably the transduction efficiency is at least 50%; more preferably at least 60%; and most preferably at least 70%.

The AAV can be any AAV serotype. Preferably, however, the AAV comprises serotype 1, 2, 6, or a mixture of two or more serotypes. In a preferred embodiment, the serotype is a mixture comprising serotypes 1 and 2.

The target hair cell or support cell to be transduced is a mammalian cell and, more preferably, a human cell. In one embodiment, the target cell is in a living mammal.

The present invention further relates to compositions for transducing a mammalian cochlear hair cell or support cell. The transduction compositions comprise an adeno-associated virus (AAV) in an amount sufficient to transduce the hair cell or support cell. The AAV comprises DNA that is exogenous to the AAV and that is typically operatively linked to a cochlear hair cell promoter or a support cell promoter.

Preferably, the AAV used in the composition is a high-titer virus of at least 10⁹ gp/μl and, more preferably, at least 10¹⁰ gp/μl. The composition may comprise one AAV serotype or a mixture of two or more. In one embodiment, the composition comprises a mixtures of at least two serotypes, for example, serotype 1 and 2.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

Further features and advantages of the invention can be ascertained from the following detailed description that is provided in connection with the illustrative drawing(s) of a preferred embodiment described below:

FIGS. 1A-F show the AAV-mediated transduction of cochlear hair cells. Cochlear explants from P1 mice were transduced with AAV-1 (FIGS. 1A-B), AAV-2 (FIGS. 1C-D) or AAV-5 (FIGS. 1E-F) carrying the CAG-GFP expression cassette. Viral transduction was determined by GFP expression (green cells; FIGS. 1A, 1C and 1E). Hair cells were identified with Myosin VI antibody and are stained red (FIGS. 1B, 1D and 1F show merged red and green images). Representative confocal images show GFP expression in both inner and outer hair cells following treatment with AAV-1 and AAV-2. Basilar regions of the cochlear explants are shown. (*) indicates GFP positive inner hair cells, (Arrow heads) indicate GFP positive outer hair cells. Magnification=100×, scale bar=8 μm. Please note that not all of the GFP positive hair cells have been marked in the figures, only a representative number for the convenience of the viewer.

FIGS. 2A-F show the transduction of E13 cochlear explants with AAV-1-CAG. The top three panels show low magnification of fluorescent images; GFP (FIG. 2A), Myosin 6 (FIG. 2B) and merge respectively (FIG. 2C) in an E13 cochlear explant cultures after 5 days in vitro. The lower three panels show high magnification of same images of GFP (FIG. 2D), myosin 6 (FIG. 2E) and merge (FIG. 2F). AAV-1-CAG is predominantly expressed in outer hair cells (OHC), but less often in hair cells (IHC) at E13.

FIGS. 3A-F show the transduction of E13 cochlear explants with AAV-2-CAG. The top three panels show low magnification fluorescent images of GFP (FIG. 3A), Myosin 6 (FIG. 3B) and merge (FIG. 3C) respectively in an E13 cochlear explant culture after 5 days in vitro. The lower three panels show high magnification images of GFP (FIG. D), myosin 6 (FIG. 3E) and merge (FIG. 3F). AAV-2-CAG predominantly expressed in outer hair cells but is also found in one inner hair cell (arrow); (*) indicates a GFP positive out hair cell.

FIGS. 4A-B show AAV-mediated transduction of support cells in murine P0 cochlear explant cultures. Representative fluorescent images of P0 cochlear explant cultures transduced with AAV-2 (FIG. 4A) or AAV-1 (FIG. 4B). Explants were transduced with 1×10¹¹ genomic particles (GP) of each AAV serotype on the day of preparation. After 5 days in culture, all explants were fixed with 4% paraformaldehyde and hair cells were labeled with anti-myosin VI antibodies. All viral vectors carried the same construct in which GFP gene expression was driven by the GFAP promoter. Green cells are GFP positive support cells. Red cells are Myosin VI positive hair cells. In FIG. 4A, (BC/IPC)=transduced border cells; (ID)=transduced interdential cells; (*)=inner hair cells; (arrowheads)=outer hair cells; (P)=transduced pillar cells; (D)=transduced Deiter's cells; and (H)=transduced Hensen's cells.

FIGS. 5A-C show the transduction of E13 cochlear explants with AAV-1-GFAP-GFP. FIGS. 5A-C show high magnification of fluorescent images of GFP (FIG. 5A), Myosin 6 (FIG. 5B) and merge (FIG. 5C), respectively, in an E13 cochlear explant after 5 days in vitro. Note the significant number of labeled cells within the sensory epithelium, but unlike the CAG promoter, the labeled cells appear to be supporting cells. (D)=transduced Deiter's cells; (P)=transduced pillar cells, (OHC)=outer hair cells; (IHC)=inner hair cells

FIGS. 6A-F show that Math1/ER induces hair cell formation in the presence of tamoxifen. Top Row: FIGS. 6A-C are low magnification images of an E14 cochlear explant that was transfected with a Math1/ER-IRES-GFP expression vector and then maintained for 6 days in vitro in the absence of tamoxifen. Hair cells in the sensory epithelium are labeled in red with an antibody against myosin 6 (FIG. 6A). Transfected cells (FIG. 6B) are green and are present in the greater epithelial ridge (GER), but no ectopic hair cells have developed. FIG. 6C shows merged red and green images of FIGS. 6A and 6B. Bottom Row: FIGS. 6D-F are low magnification images of a sister explant that was transfected with the same vector but was maintained in media with 15 nM tamoxifen for the duration of the experiment. Hair cells labeled with an antibody against myosin 6 are shown in FIG. 6D. FIG. 6E shows the transfected cells and FIG. 6F is the overlay of FIGS. 6D and 6E. In addition to the row of hair cells within the sensory epithelium, numerous ectopic hair cells are also present in the GER (arrowheads). The region of ectopic hair cells correlates exactly with the region of transfected cells (arrows) and double-labeled cells can be identified in the merged image (arrows). Scale bar equals 200 microns.

FIGS. 7A-F show the GFP expression following in vivo transduction of the murine cochlea with AAV-1-CAG-GFP. SV=scala vestibuli; SM=scala media; ST=scala tympani; RM=Reissners' membrane; L=limbus; SL=spiral igament; SG=spiral ganglion (arrow); (*) tunnel of corti; Inner hair cells=arrows. Representative fluorescent images of paraffin embedded cochlea stained with anti-GFP antibody. 1×10⁹ genomic particles of AAV-1-CAG-GFP virus was injected directly into the cochlea of 4 month old CD1 mice via cochleostomy. Mice were sacrificed after 4 weeks. Cochlea were fixed with 4% paraformaldehyde and paraffin embedded. 10 μm thick sections were stained with fluorescent tagged anti-GFP antibody (bright green cells). In FIG. A, GFP positive cells are observed in cells lining the scala tympani and scala vestibuli. The transduced cells are found within the scala media in 2 out of the 5 animals examined (See FIGS. 7B-C). Specifically, transduction is observed within hair cells, support cells and spiral ganglion cells. FIG. 7D is a high magnification image of FIG. 7B. FIGS. 7E-F are high magnification images of FIG. 7C. Note the presence of GFP positive cells within what appears to be hair cells and support cells.

FIGS. 8A-B shows AAV can be used to successfully transduce cochlear hair cells and support cells in vivo. AAV-2 vectors carrying the green fluorescent protein (GFP) gene under control of the glial fibrilary acidic protein (GFAP) promoter were delivered directly to the basal turn of the cochlea via the scala tympani of the guinea pig. Transduction was confirmed by immunocytochemical analysis using anti-GFP antibodies and DAB. Representative images of whole mounts prepared from injected (FIG. 8A) and control (FIG. 8B) cochlea. The region of strong GFP staining in FIG. 8A (indicated by arrows) is absent in the control, uninjected cochlea. The strongest region of GFP expression appears to be in the support cells, between the inner and outer rows of hair cells and possibly beneath the hair cells. This is consistent with our previous data and suggests that GFP expression from the GFAP promoter is localized to support cells and not hair cells.

FIGS. 9A-B shows AAV-2 mediated transduction of support cells within the guinea pig cochlea. To confirm that the GFAP promoter selectively drives transgene expression within support cells, cross sections of paraffin embedded cochleas from control were examined (FIG. 9A) and AAV-2-GFAP-GFP injected (FIG. 9B) guinea pigs. GFP expression was visualized by immunocytochemical analysis using DAB. No GFP specific staining was detected in the control cochleas. However, GFP positive cells were observed in the AAV-2-GFAP-GFP injected animals. Based on the morphology and localization of the DAB positive cells (see FIG. 10), it is clear that GFAP selectively drives expression of transgenes within the support cells of the cochlea and that AAV-2 is an efficient means of gene delivering transgenes to these cells. Note, the strongest GFP expression observed with the GFAP promoter was in pillar cells, with moderate expression in border, inner phalangeal and Deiter's cells.

FIG. 10 is a schematic diagram of the mammalian cochlea: (1) baselar membrane; (2) Hensen's cells; (3) Deiter's cells; (4) outer hair cells; (5) outer pillar cells; (6) inner pillar cells;,(7) outer hair cell; (8) inner phalangeal cells; (9) border cell; (10-11) interdential cells.

### DETAILED DESCRIPTION OF THE PREFERRED EMBODIMENTS

All patents and literature references cited in this specification are hereby incorporated by reference in their entirety. As used in the specification and in the claims, “a” can mean one or more, depending upon the context in which it is used.

The present invention provides a method of transducing mammalian cochlear cells, preferably hair cells or support cells. The method comprises the step of delivering an adeno-associated virus (AAV) to a target hair cell or support cell. The AAV used to transduce the cochlear cell is modified and comprises DNA that is exogenous to the AAV and operatively linked to a promoter.

A cochlear “hair cell” is a sensory cell in the ear. A normal hair cell is in synaptic contact with sensory as well as efferent fibers of the auditory nerve and has fine projections resembling hairs. Hair cells are also sometimes referred to as Corti's cells. There are two types of hair cells: outer and inner hair cells. Outer hair cells are distal from the spiral limbus, and generally there are three to five rows of hair cells that run the length of the cochlear duct (about 20,000 in number in humans). Inner hair cells are proximal to the spiral limbus. There is only one row of inner hair cells that run the length of the cochlear duct (about 3500 in number in humans).

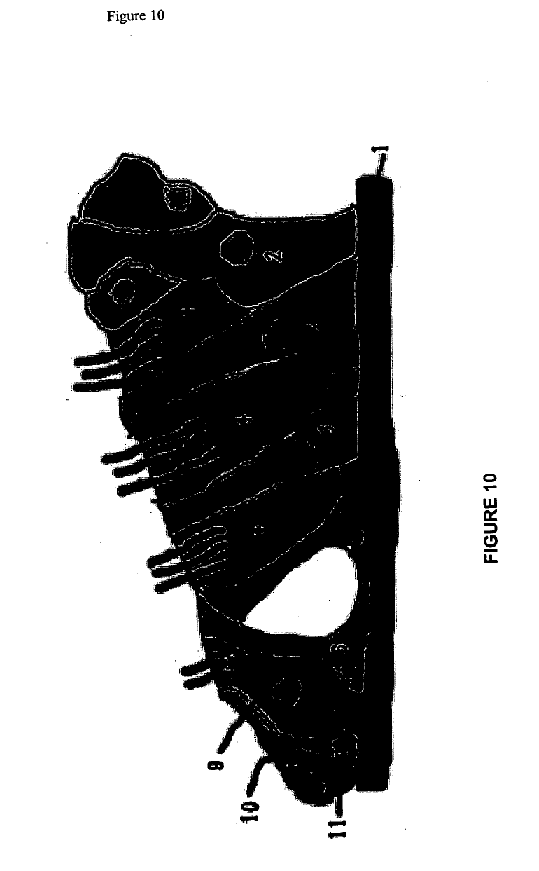

A cochlear “support cell” is located in the sensory epithelium and is in close contact with a cochlear hair cell, preferably direct contact. Generally, support cells include: Dieter's cells, Hensen's cells, pillar cells, inner phalangeal cells, border cells, or interdential cells. See, FIG. 10 for a schematic diagram of the mammalian cochlea.

The term “transduction” denotes the delivery of a DNA molecule to a recipient cell either in vivo or in vitro, via AAV.

AAV: Eight different AAV serotypes (AAV-1-8) have been identified based on amino acid sequence differences in their respective capsid proteins. Serotypes 1 and 6 share >99% amino acid homology and therefore are not functionally differentiated. The AAV used in the present invention can be any AAV serotype. In a preferred embodiment, however, the serotype comprises serotype 1, 2, 6, or a mixture of two or more serotypes, for example, a mixture comprising serotypes 1 and 2.

The AAV used in the present invention is a derivative of the adeno-associated virus, into which exogenous DNA has been introduced. The construction of infectious recombinant AAV and methods of purification are well known in the art. See, e.g., U.S. Pat. Nos. 5,173,414; 5,139,941; 5,741,683; 6,458,587; 6,475,769; and 6,783,972; Zolotukhin et al. (1999); and Grimm et al. (1998 and 1999), all of which are incorporated herein by reference.

The AAV genome is composed of a linear, single-stranded DNA molecule that contains 4681 bases (Berns and Bohenzky, supra). The genome includes inverted terminal repeats (ITRs) at each end that function in cis as origins of DNA replication and as packaging signals for the virus. The ITRs are approximately 145 bp in length. The internal nonrepeated portion of the genome includes two large open reading frames, known as the AAV rep and cap regions, respectively. These regions code for the viral proteins that provide AAV helper functions, i.e., the proteins involved in replication and packaging of the virion. Specifically, a family of at least four viral proteins is synthesized from the AAV rep region, Rep 78, Rep 68, Rep 52 and Rep 40, named according to their apparent molecular weight. The AAV cap region encodes at least three proteins, VP1, VP2 and VP3. For a detailed description of the AAV genome, see, e.g., Muzyczka (1992).

AAV vectors can be engineered to carry an exogeneous nucleotide sequence of interest (e.g., a selected gene—e.g., Hath1 or SOX2, antisense nucleic acid molecule, or the like) by deleting, in whole or in part, the internal portion of the AAV genome and inserting the DNA sequence of interest between the ITRs. The ITRs remain functional in such vectors allowing replication and packaging of the AAV containing the heterologous nucleotide sequence of interest. The heterologous nucleotide sequence is also typically linked to a promoter sequence capable of driving gene expression in the patient's target cells under the certain conditions. Termination signals, such as polyadenylation sites, can also be included in the vector.

For propagation of the vector in vitro, susceptible cells are co-transfected with the AAV-derived vector and a suitable AAV-derived helper virus or plasmid. Preferably, the vector retains from AAV essentially only the recognition signals for replication and packaging.

The AAV-derived sequences do not necessarily have to correspond exactly with their wild-type prototypes. For example, the AAV vectors of the present invention may feature mutated inverted terminal repeats, etc., provided that the vector can still be replicated and packaged with the assistance of helper virus, and still infect target cells. Optionally, the helper virus may be removed in those embodiments where a helper virus is used, for example, by heat inactivation at 56° C. for 30 minutes, or separated from packaged AAV vectors by centrifugation in a cesium chloride gradient.

In one embodiment the AAV is produced using the plasmid-based method as described in Lai, et al., (2002) and the virus is purified on iodixanol gradients as described in Zolotukhin et al., (2002).

Viruses are titered by the number of genomic particles per ml. Titers of the AAV can vary, particularly depending upon the target cell, but preferably the AAV used is a high-titer virus of at least 10⁹ gp/μl and more preferably, at least 10¹⁰ gp/μl. Methods of producing high-titer viruses are also well known in the art. See, e.g., U.S. Pat. No. 6,632,670 (teach methods of generating high-titer, contaminant free, recombinant AAV vectors in large quantities).

DNA: By “exogenous DNA” is meant any heterologous DNA, i.e., not normally found in wild-type AAV that can be inserted into the AAV for transfer into the target cell. By “operatively linked” is meant that the promoter can drive expression of the exogenous DNA, as is known in the art, and can include the appropriate orientation of the promoter relative to the exogenous DNA. Furthermore, the exogenous DNA preferably has all appropriate sequences for expression. The DNA can include, for example, expression control sequences, such as an enhancer, and necessary information processing sites. Typically, because of the packaging limitations of AAV, the exogenous DNA will have a length of about 10-5,000 bases. Preferably, the DNA is 100 to 4,000 bases.

Preferred examples include DNA that encodes a protein that promotes cochlear hair cell growth or cell differentiation, e.g., promotes support cell differentiation into hair cells, or corrects a genetic mutation. Non-limiting examples include DNA that encodes Math1, Hath1, SOX2, connexin 26, or a growth factor protein. Examples of growth factor proteins include: Nerve Growth Factor (NGF), Glial-Derived Neurotrophic Factor (GDNF), and Fibroblast Growth Factor (FGF).

In a preferred embodiment, the DNA encodes Math1 or the human ortholog, Hath1. Expression of the basic helix-loop-helix transcription factor, Math1 or Hath 1, has been shown to be both necessary and sufficient for hair cell differentiation. Zheng and Gao, (2000) reported the development of myosin VIIa positive cells expressing Math1 within the greater epithelial ridge following the electroporation of rat cochlear explants with the Math1 gene. More recently, Kawamoto et al. (2003) also observed the limited appearance of immature hair cells within the organ of corti and nonsensory epithelial cells following Adenovirus-mediated delivery of the Math1 gene to the scala media. Interestingly, Kawamoto et al. also reported that axons were extended to some of the immature hair cells within the nonsensory regions. During development, Math1 is only expressed between E12.5-PO (Zuo, 2002).

In another preferred embodiment, the DNA encodes an ER receptor fusion protein, such as, Math1/ER, Hath1/ER, or SOX2/ER protein. In this embodiment, the fusion protein is useful in regulating the activity of the Math1, Hath1, or SOX2 proteins. Activity is regulated by the cytoplasmic retention of the fusion protein by the estrogen receptor. The use of an activating compound, e.g., tamoxifen, is necessary to permit translocation of the fusion protein to the nucleus where it is then functionally active. In one embodiment, tamoxifen is administered at least a day after transduction and, more preferably, several weeks after transduction. In a preferred embodiment, the activating compound is administered two to three weeks after transduction. By waiting to administer the activating compound, the patient is allowed to recover from treatment and a high and consistent level of exogenous gene expression in the patient is more likely.

Promoter: The promoter can be any desired promoter, selected by known considerations, such as the level of expression of the DNA operatively linked to the promoter and the cell type in which the DNA is to be expressed, e.g., hair cells or support cells. Promoters can be an exogenous or an endogenous promoter. Promoters can be prokaryotic, eukaryotic, fungal, nuclear, mitochondrial, viral, etc. Additionally, chimeric regulatory promoters for targeted gene expression can be utilized. Preferred promoters include cochlear hair specific promoters and support cell specific promoters.

Boeda et al. (2001) recently characterized the MYO7A promoter, which exhibits strong, selective expression in hair cells of the cochlea and vestibule. More recently, the glial fibrillary acid protein (GFAP) promoter was shown to have selective activity within certain subpopulations of support cells (R10 et al., 2002). The authors observed GFAP activity in all supporting cells early after birth with an intensity gradient decreasing from the base towards the apex. Likewise, our laboratory has observed a similar pattern of GFP expression in P0 cochlear explants when the GFAP promoter was used to drive transgene expression. Interestingly, R10 et al, also reported that after P15 GFAP expression was mostly restricted to inner phalangeal cells, border cells and Dieter's cells.

Examples of others promoters that can be used include the murine CMV (mCMV) promoter, which exhibits selectivity for astrocytes (Aiba-Masago et al., 1999). Furness and Lawton (2003) reported that the astrocytic glutamate transporter (GLAST) is expressed only in border cells and inner phalangeal cells of mature guinea pigs. Indeed, a similar pattern of GLAST expression was observed in P0 cochlear explants cultures. Other examples include Jagged-1 and Notch 1, which may also be useful for support cell specific expression.

Examples of preferred support cell specific promoters include: the glial fibrillary acidic protein (GFAP) promoter, the excitatory amino acid transporter-1 (EAAT1) promoter, the GLAST promoter and the murine cytomegalovirus (mCMV) promoter. Examples of preferred hair cell specific promoters include: the human cytomegalovirus (CMV) promoter, the chicken β-actin/CMV hybrid (CAG) promoter, and the myosin VIIA promoter. In one embodiment, the preferred promoter is the CAG promoter.

Delivery: Delivery can be accomplished by any standard means for administering AAV. For example, by simply contacting the AAV, optionally contained in a desired liquid such as tissue culture medium, or a buffered saline solution, with the target cell. The AAV can be allowed to remain in contact with the target cell for any desired length of time, and typically the AAV is administered and allowed to remain for a time sufficient to effectively transduce the target cell.

For in vivo delivery, the AAV may be delivered by any suitable method. Examples of delivery methods that can be used include: via osmotic mini-pump infusion via the round window (Derby et al., 1999, Komeda et al., 1999; Raphael et al., 1996; and Yagi et al 1999); delivery into the scala tympani either via the round window or cochleostomy (Carvalho and Lalwani, 1999; Han et al., 1999; Jero et al., 2001; Lalwani et al., 1996, 1997, 1998a, 1998b; Luebke et al., 2001b; Raphael 2001; Waring et al., 1999); via direct injection of virus into the endolymphatic sac (Yamasoba et al. (1999)); and via direct injection of virus into the scala media (Ishimoto et al. (2002). In a preferred embodiment, the AAV is delivered directly or indirectly into the cochlea. In one embodiment the AAV is delivered directly into the scala tympani.

Appropriate doses will depend on the subject being treated (e.g., human or nonhuman primate or other mammal), age and general condition of the subject to be treated, the severity of the condition being treated, the mode of administration of the AAV, among other factors. An appropriate effective amount can be readily determined by one of skill in the art.

Thus, a “therapeutically effective amount” will fall in a relatively broad range that can be determined through clinical trials. For example, for in vivo injection, i.e., injection directly to the subject, a preferred therapeutically effective dose will be on the order of from 10 μl to 500 μl of an AAV titer of 10¹⁰ gp/μl. In some subjects it will preferably be 50 μl to 150 μl, for example, in one embodiment 100 μl is delivered to the human cochlea.

AAV compositions: The present invention also relates to compositions for transducing a mammalian cochlear hair cell or support cell. The transduction compositions comprise an adeno-associated virus (AAV) in an amount sufficient to transduce the cochlear hair cell or support cell. The AAV comprises DNA that is exogenous to the AAV and that is typically operatively linked to a cochlear hair cell promoter or a support cell promoter.

The AAV compositions will comprise sufficient AAV to effectively produce a therapeutically effective amount of the protein of interest in the target cells, i.e., an amount sufficient to reduce or ameliorate symptoms of the disease state in question or an amount sufficient to confer the desired benefit. Thus, the AAV will be present in the composition in an amount sufficient to provide a therapeutic effect when given in one or more doses.

The composition may also contain a other ingredients, such as, pharmaceutically acceptable excipients. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in REMINGTON'S PHARMACEUTICAL SCIENCES (Mack Pub. Co., N.J. 1991).

Preferred excipients confer a protective effect on the AAV such that loss of AAV, as well as the loss of transduceability resulting from formulation procedures, packaging, storage, transport, and the like, is minimized.

The present invention is more particularly described in the following examples that are intended as illustrative only since numerous modifications and variations therein will be apparent to those skilled in the art.

### EXAMPLES

### Example 1

### Transduction of Cochlear Hair Cells and Support Cells in vitro

Materials and Methods

Plasmid construction. Two separate trans-gene plasmids were constructed for packaging into AAV, containing either the CAG promoter or the GFAP promoter. The CAG promoter is a ubiquitous promoter and has been shown to drive robust expression in liver and brain (Xu et al., 2001; Klein, et al., 2002). A 730-bp BamHI-EcoRI fragment containing the humanized Renilla green fluorescent protein (hrGFP) gene (Stratagene) was subcloned into the CAG or GFAP vector. Each construct also contained a 3′ WPRE. The WPRE evolved to promote the expression of intronless viral messages and has been shown to increase the stability and level of gene expression, both in vitro and in vivo (Klein, et al., 2002; Loeb, et al. 1999).

Virus production. AAV serotypes 1, 2, and 5 were packaged in HEK293T cells. Cultures were maintained in growth medium consisting of DMEM supplemented with 10% FBS, 0.05% penicillin/streptomycin (5000 U/ml), 0.1 mM MEM nonessential amino acids, 1 mM MEM sodium pyruvate, and gentamicin (25 mg/ml). The day before transfection approximately 1.5×10⁷ cells were plated on 150-mm dishes containing growth medium. Twenty-four hours later, medium was changed to DMEM containing 5% FBS and antibiotics and cells were transfected using Polyfect transfection reagent (Qiagen). The plasmid-based method of AAV production was used (Lai, et al., 2002). Plasmids used for transfection were (1) pFA6 (adenoviral helper plasmid); (2) pRVI (cap and rep genes for AAV serotype 2), pH21 (cap gene for AAV serotype 1 and rep gene for serotype 2), or pH25a (cap gene for AAV serotype 5 and rep gene for serotype 2); and (3) trans-gene plasmid containing the GFP expression cassette flanked by the AAV-2 ITRs. These plasmids were obtained from the laboratory of Dr. Matthew During Jr. (University of Auckland, New Zealand). Virus was purified on iodixanol gradients as previously described (Zolotukhin et al., 2002). Virus titers were determined by quantitative PCR and expressed in genomic particles/ml.

Cochlear cultures. Primary cochlear explants were prepared from E13 or P0-P1 CD₁ mice (Charles River). The day of birth was designated postnatal day 0. All animal procedures were performed in strict accordance with the NTH Guide for Care and Use of Laboratory Animals and were approved by the University of Montana Institutional Animal Care and Use Committee. Cochleas were dissected as described previously (Mueller et al., 2002; Raz et al. 1999). Briefly, the cochlea and vestibular region were aseptically dissected away from the skull in cold dissection medium composed of 1×HBSS containing 5 mM Hepes and 0.6% glucose. The vestibular region was pinned down and the bony outer capsule was carefully dissected away from the rest of the cochlea. Since the cochlea exceeds more than one turn at P0, the cochlea was cut into two pieces and carefully transferred to Mat-Tek dishes coated with 0.05 mg/ml poly-D-lysine (BD) followed by 3.75% Matrigel (BD). Culture medium was DMEM supplemented with 10% FBS, N2 (1:100; Invitrogen), Penicillin G (1500 U/ml; CalBiochem), and Fungizone (9 μg/ml; Calbiochem). AAV-1-CAG-hrGFP, AAV-2-CAG-hrGFP, AAV-5-CAG-hrGFP, AAV-1-GFAP-hrGFP, AAV-2-GFAP-hrGFP, or AAV-5-GFAP-hrGFP was added to the medium for a final concentration of 10¹⁰ gp per dish at the time of plating. Cultures were maintained at 37° C., 5% CO₂ for 5 days in a humidified incubator.

Immunocytochemistry. After 5 days, medium was carefully aspirated and cultures were fixed in cold 4% paraformaldehyde (PEA) at room temperature for 30 min followed by a postfix in ice-cold methanol for 2 min at −20° C. To remove residual PEA and methanol, cultures were rinsed with PBS (3×15 min). Tissue was permeabilized with PBS plus 0.5% Triton X (PBS-Tx) for 1 h at room temperature. Tissue was then blocked with 5% normal goat serum (NGS) for 1 h at room temperature. All immunocytochemical reagents were diluted in PBS-Tx unless otherwise noted.

All primary antibodies were diluted in PBS-Tx containing 1% NGS and were incubated overnight at 4° C. Anti-myosin VI (Sigma) was used to identify hair cells. Unbound primary antibody was removed by washing tissue with PBS-Tx (3×20 min). Primary antibodies were detected by incubation with secondary Alexa-Fluor 546 (1:2000; Molecular Probes) for 1 h at room temperature. To remove any unbound secondary antibody and residual salts, tissues were washed with PBS-Tx (3×10 min) and PBS (2×5 min). Embryonic culture images were taken on a Zeiss LSM510 confocal microscope and PI cultures were imaged on a Bio-Rod Radiance 2000 MP laser scanning confocal microscope. Z stack images were merged and analyzed using MetaMorph software (Universal Imaging). Positive identification of transduced cells was determined by GFP green fluorescence at 488 nm.

The specific cell types were identified by red immunofluorescence at 543 nm. Cell counts were expressed as a ratio of green GFP expression over the cell-specific red immunolabel. Three to four fields, with a total length of approximately 250 μm of apex or base, were counted at 40× magnification. Preliminary experiments revealed a difference in transduction efficiency between inner and outer hair cells so separate counts were obtained for each. Counts were also obtained for apical and basal portions of the cochlea due to the fact that transduction efficiency appears to follow a basal-to-apical preference. GFP gene expression levels in inner and outer hair cells were determined by measuring the relative mean fluorescence density (total fluorescence/area of cell) using Image Pro Plus software (Media Cybernetics, Inc., Silver Springs, Md., USA). Data were analyzed with ANOVA using GraphPad Instat. A P value<0.05 was considered statistically significant.

Results

A. AA V Transduce Hair Cells in Murine Cochlear Explants

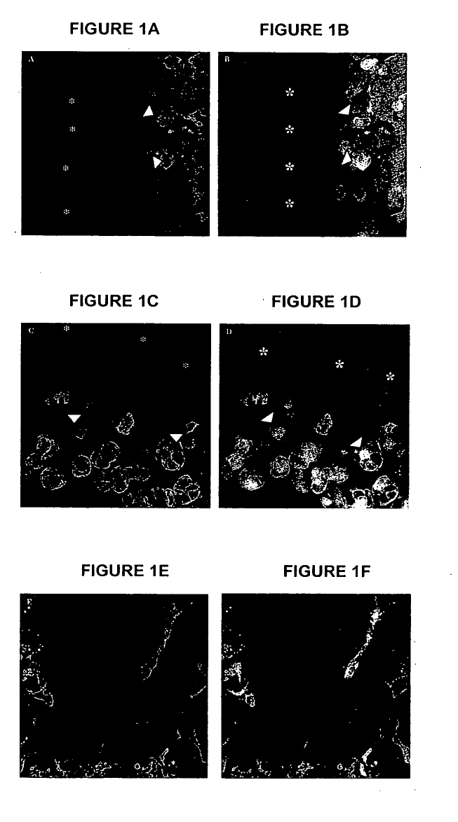

The ability of AAV serotypes 1, 2 and 5 to transduce hair cells within cochlear explants of PO-1 mice was studied. Previous studies had indicated that AAV was not capable of transducing cochlear hair cells due to the lack of heparin sulfate on their cell surface. (Jero et al., 2001; Kho et al., 2000; Luebke et al., 2001b). In contrast to the prior art teachings, the inventors surprisingly discovered that, using their method, AAV is in fact capable of transducing hair cells, inner and outer. See, FIGS. 1-3.

As shown in FIGS. 1A-F, cochlear hair cells were successfully transduced with AAV-1 (FIGS. 1A-B), AAV-2 (FIGS. 1C-D), and AAV-5 (FIGS. 1E-F) carrying the CAG-GFP expression cassette. Although slight differences in the numbers of inner hair cells and outer hair cells transduced by AAV-1 compared with AAV-2 within the basal region were observed, these differences were not statistically significant (Table 1). Approximately 43% of inner hair cells and 64% of outer hair cells were transduced by AAV-1 compared to 36% of inner hair cells and 59% of outer hair cells transduced with AAV-2.

TABLE 1
Transduction efficiency and GFP expression levels

| | | | | |
|---|---|---|---|---|
| Serotype | IHC base | OHC base | IHC apex | OHC apex |

Transduction efficiency

| | | | | | | | | |
|---|---|---|---|---|---|---|---|---|
| AAV-1 | 108/253 | (43%) | 257/402 | (64%) | 34/180 | (19%) | 433/660 | (66%) |
| AAV-2 | 42/118 | (36%) | 211/357 | (59%) | 25/159 | (16%) | 152/408 | (37%) |
| AAV-5 | 2/279 | (0.7%) | 6/642 | (0.9%) | 0/167 | (0%) | 5/405 | (1%) |

GFP expression levels

| AAV-1 | 11.21 ± 1.0 | 30.39 ± 1.91 | 14.39 ± 2.54 | 33.96 ± 2.52 |
|---|---|---|---|---|
| AAV-2 | 7.29 ± 0.69 | 35.08 ± 4.62 | 9.91 ± 1.59 | 21.44 ± 4.87 |
| Transduction efficiency shows the percentages of hair cells transduced by each AAV serotype. Values are presented as the total number of myosin VI-positive hair cells counted and represent cell counts from a minimum of four different experiments. Values are given for each serotype for both basal and apical | | | | |
| # regions. GFP expression levels show the relative GFP expression levels observed in inner hair cells and outer hair cells in the base and apex of the cochlea. Values were determined by measuring the relative fluorescence intensity/area of individual cells. IHC, inner hair cell; OHC, outer hair cell. | | | | |

Robust GFP expression in outer hair cells transduced with either AAV-1 or AAV-2 was observed. However, GFP expression was lower (P<0.001) in inner hair cells transduced with AAV-1 and AAV-2 (Table 1). Comparable low levels of GFP expression within the inner hair cells of the base and apex following transduction with either AAV-1 or AAV-2 were detected. The major differences between AAV-1 and AAV-2 were observed within the outer hair cells of the apical region. Lower GFP expression was detected in the apical outer hair cells transduced with AAV-2 compared to AAV-1 (Table 1).

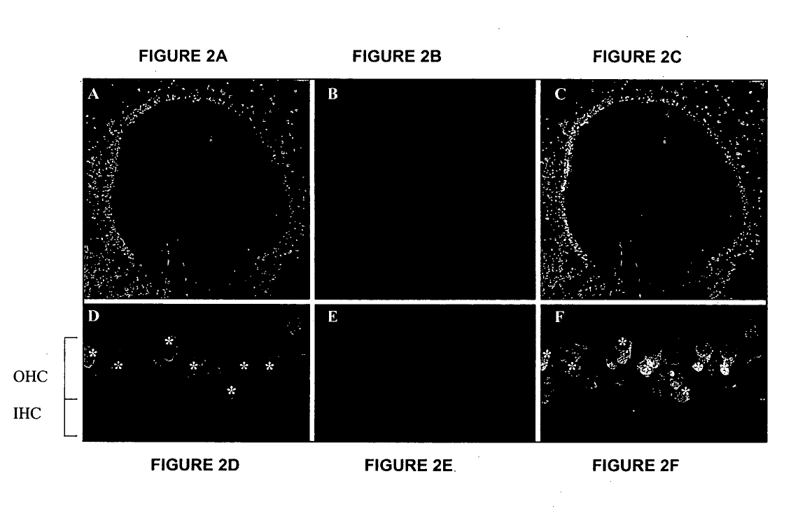

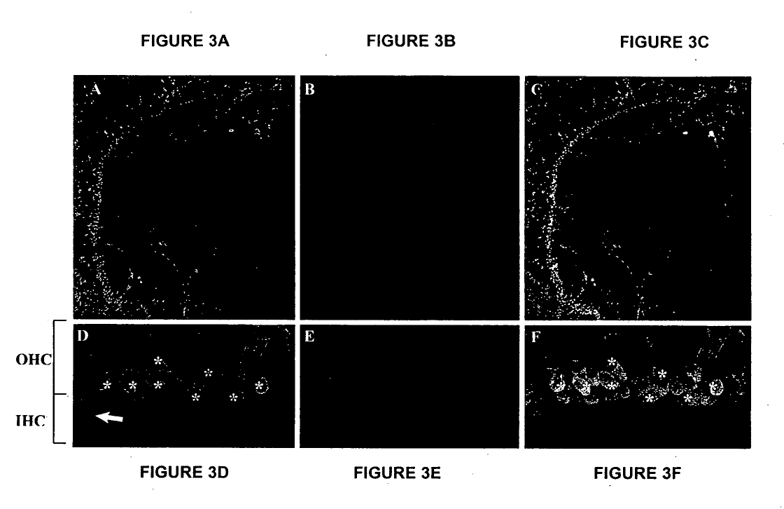

Given the potential that developmental or age-dependent modifications can affect the expression and regulation of transcriptional activators as well as the cell surface expression of target receptors, a qualitative analysis was performed in which E13 cochlear explants were transduced with AAV-1 and AAV-2 carrying the CAG-GFP construct. See, FIGS. 2-3. FIGS. 2A-F show images of E13 cochlear explants transduced with AAV-1-CAG; whereas, FIGS. 3A-F show representative images of E13 cochlear explants transduced by AAV-2-CAG. Similar to the postnatal day 0 (P0) explants, robust GFP expression was observed in the outer hair cell population following transduction with both AAV-1 and AAV-2. However, limited GFP expression in the inner hair cells of E13 explants compared to P0 cultures was observed. As with the P0 cultures, a clear gradient of GFP expression was observed in the transduced E13 explant cultures, with the strongest expression detected in the base and decreasing toward the apex.

These results clearly demonstrate that both AAV-1 and AAV-2 can transduce cochlear hair cells and that the CAG promoter is functionally active in these cells. It is also clear from these results that AAV-5 can mediate transduction of murine cochlear hair cells, although not as efficiently as AAV-1 and AAV-2.

B. AAV Transduction of Murine Support Cells in Cochlear Explants

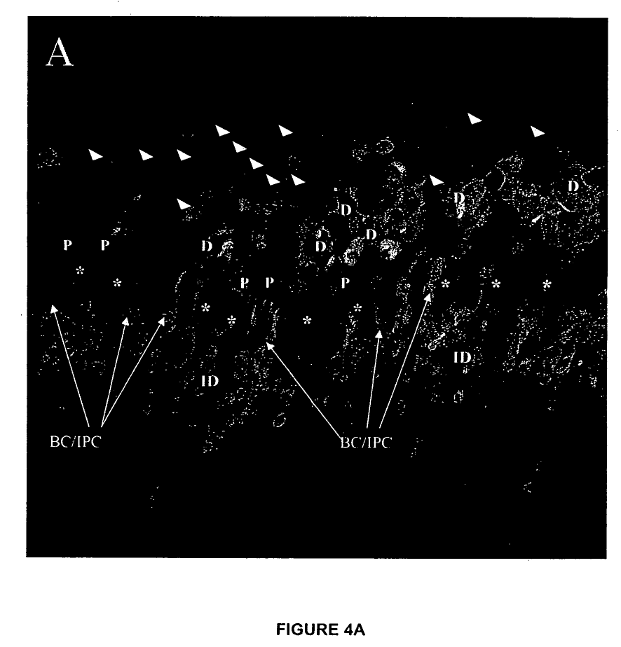

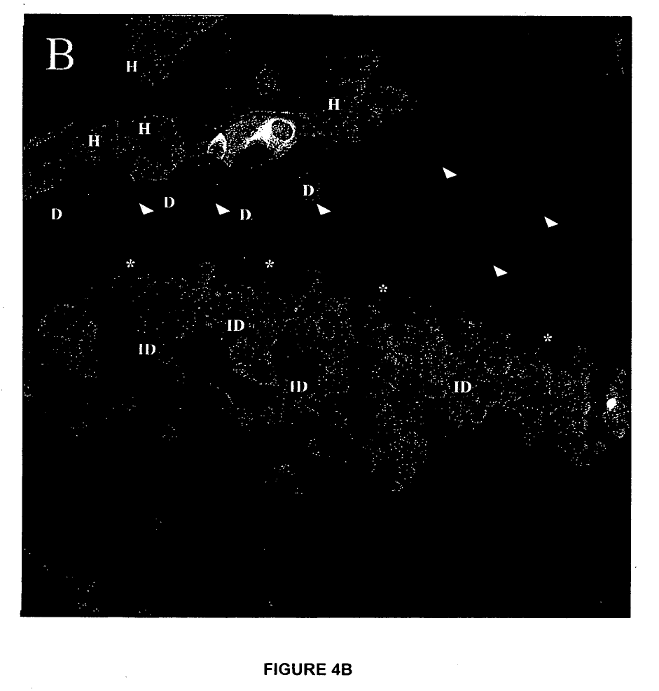

To examine AAV-mediated transduction of support cells within murine cochlear explants, a second expression cassette in which the GFP gene was placed under the control of the astrocyte-specific GFAP promoter was created. The GFAP promoter was previously reported to be active in all support cell populations of newborn guinea pigs (R10 et al., 2002). As FIG. 4 indicates, robust transduction of support cells with both AAV-1 and AAV-2 was observed. GFP expression following transduction with the AAV-5 vector was limited in comparison to transduction with AAV-1 and AAV-2. Using the GFAP promoter, GFP expression was observed in hair cells with any of the serotypes examined.

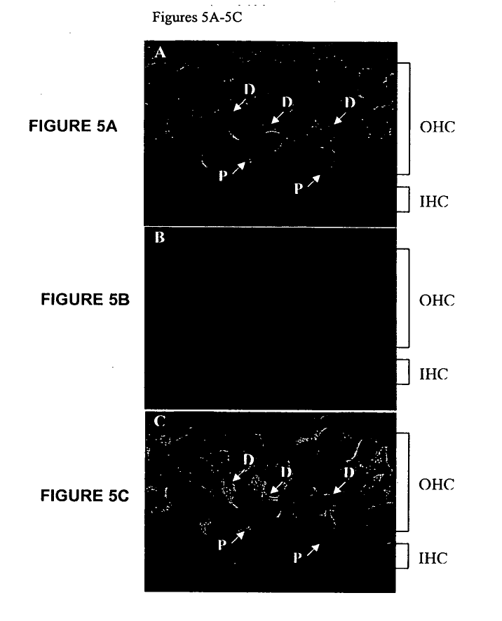

Due to the lack of good support cell-specific antibodies, specific support cell populations were identified by morphology and location. This precluded a quantitative analysis from being completed in order to determine the actual percentage of transduced support cells. Based on localization in relation to the inner and outer hair cells, however, strong GFP expression was observed in the Hensen's cells, Dieter's cells, pillar cells, inner phalangeal cells, border cells and interdential cells following transduction of P0 cochlear explants with AAV-2 (FIG. 4A). Likewise, transduction of P0 explants with AAV-1 also lead to robust expression in support cells, primarily in the Hensen's cells and interdential cells flanking the inner and outer hair cells (FIG. 4B). AAV-1 was also shown to strongly transduce E13 support cells (FIG. 5). GFP expression was more limited in support cells transduced with AAV-5

Thus, as evidenced by FIGS. 4-5, AAV efficiently transduces support cell populations, especially AAV-1 and AAV-2. The differences in cellular tropisms between AAV-1 and AAV-2 could be beneficial for certain treatments.

### Example 2

### Inducible Activation of Math/ER Fusion Protein

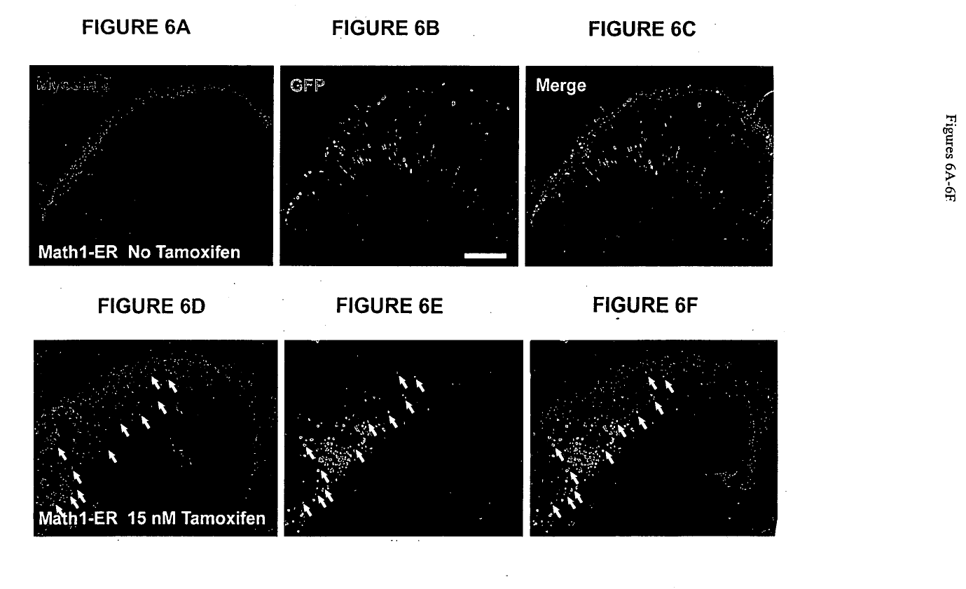

We were interested in developing an inducible Math1 protein that would permit tighter regulation of Math1 activity and allow the study of the temporal effects of transient Math1 activity on hair cell development. Therefore, a Math1/estrogen receptor (ER) construct was cloned and expressed under control of the CMV promoter. In the absence of an inducible or activating agent, such as tamoxifen, the Math1/ER fusion protein is sequestered in the cytoplasm. In the presence tamoxifen, the fusion protein is trafficked to the nucleus where Math1 can activate its target sequences and induce hair cell differentiation. FIG. 6 shows fluorescent images illustrating this point. E14 mouse cochlea were transfected by electroporation with a Math1/ER-IRES-GFP construct. Cultures were treated for 6 days with 15 nM tamoxifen. Control sister cultures were maintained in tamoxifen-free media. After 6 days, cultures were fixed and stained for the hair cell-specific marker myosin 6. As shown by FIG. 6, by fusing Math1 with the ER protein it is possible to regulate the activity of Math1. This provides the ability to control hair cell differentiation after transduction has occurred.

### Example 3

### Transduction of Cochlear Hair Cells and Support Cells in vivo via Direct Injection by Cochleostomy

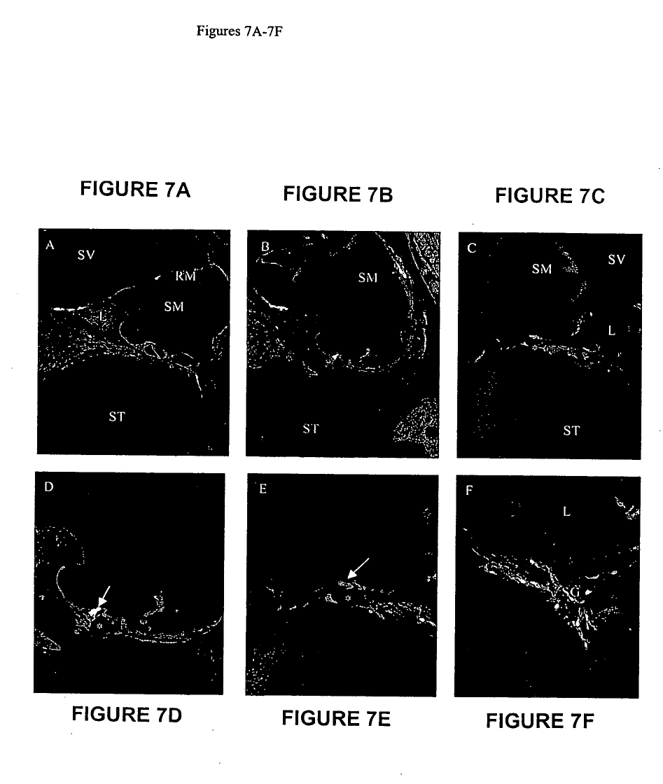

The direct injection of AAV-1-CAG-GFP into the murine cochlea via a cochleostomy resulted primarily in the transduction of cells associated with the paralymphatic space. As FIG. 7A shows, GFP positive cells are observed in cells lining the scala tympani and scala vestibuli. However, transduced cells were found within the scala media in 2 out of the 5 animals examined (FIGS. 7B-C). Specifically, transduction was observed within hair cells, support cells and spiral ganglion cells. These results demonstrate that AAV mediated transduction of hair cells and support cells within the organ of corti is an effective approach to gene transfer. The limited transduction efficiency within cells of the organ of corti is in line with previously published results and suggests that virus entry into the scala media only occurs if the paralymphatic-endolymphatic barrier is breeched. Thus, direct delivery of viral vectors to the endolymphatic space will greatly improve transduction efficiency within the organ of corti.

### Example 4

### Transduction of Cochlear Support Cells in vivo via Direct Injection into the Scala Tympani

Recombinant AAV-2 GFAP-GFP was injected directly into the scala tympani of adult male guinea pigs (approximately 300 g) according to the method of Luebke et al. (2001b). Briefly, the inferior wall of the tympanic bony bulla was surgically exposed and a small hole made in the bony bulla. A small hole was drilled into the scala tympany of the basal turn of the cochlea just below the oval window. Ten μl of virus in an artificial parilymph solution were delivered to the scala tympani via a microcannula attached to a microinfusion osmotic pump. The entire solution was delivered at a rate of 500 nl/hour. The bony defect in the bulla was closed using Duralon and the incision closed with Dermalon.

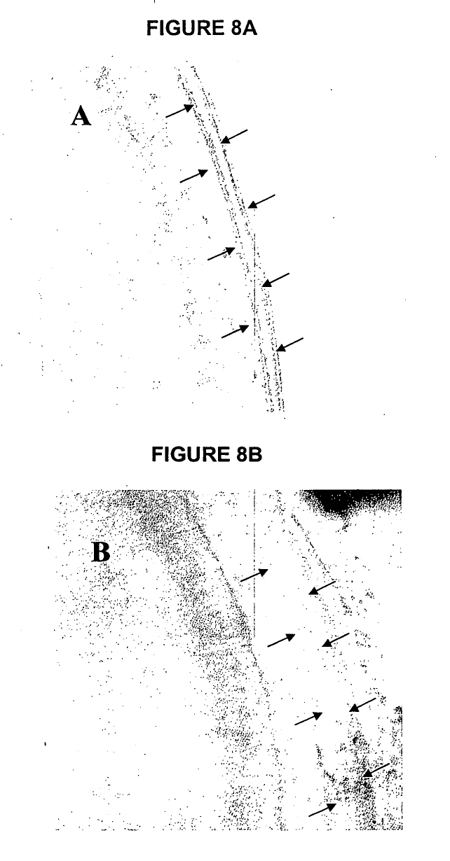

As shown in FIGS. 8A-B, AAV successfully transduced support cells in vivo—Dieter's cell, Hensen's cell, pillar cell, inner phalangeal cell, border cell, or interdential cell. AAV-2 vectors carrying the green fluorescent protein (GFP) gene under control of the GFAP promoter were delivered directly to the basal turn of the cochlea via the scala tympani of the guinea pig. Transduction of the support cells was confirmed by immunocytochemical analysis using anti-GFP antibodies and DAB. Representative images of whole mounts were prepared from injected (FIG. 8A) and control (FIG. 8B) cochlea. The region of strong GFP staining in FIG. 8A (indicated by arrows) is absent in the control, uninjected cochlea. As can be seen, the strongest region of GFP expression is in the support cells, between the inner and outer rows of hair cells and possibly beneath the hair cells.

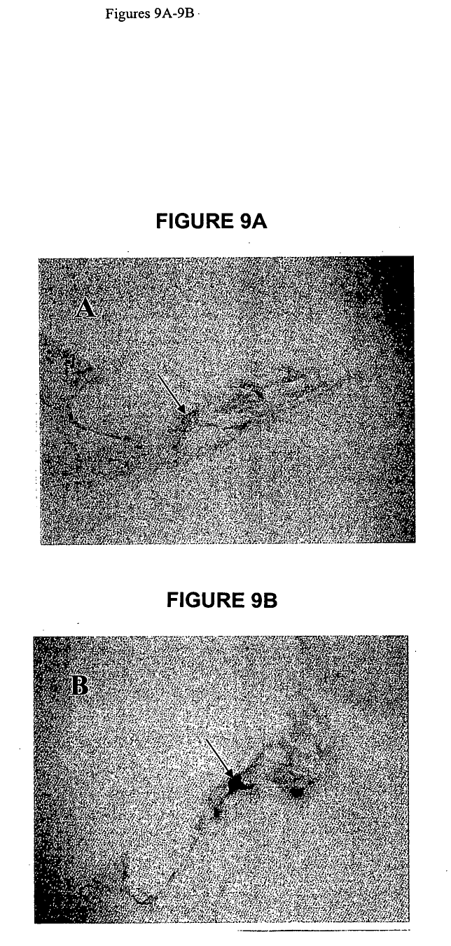

In addition, to confirm that GFAP promoter selectively drives transgene expression within support cells, cross sections of paraffin embedded cochleas from the control were examined (FIG. 9A) and AAV-2-GFAP-GFP injected (FIG. 9B) guinea pigs. GFP expression was visualized by immunocytochemical analysis using DAB. No GFP specific staining was detected in the control cochleas. However, GFP positive cells were observed in the AAV-2-GFAP-GFP injected animals. Based on the morphology and localization of the DAB positive cells, it is clear that GFAP selectively drives expression of transgenes within the support cells of the cochlea and that AAV-2 is an efficient means of gene delivering transgenes to these cells.

### REFERENCES CITED

Aiba-Masago, S., S. Baba, et al. (1999). “Murine cytomegalovirus immediate-early promoter directs astrocyte-specific expression in transgenic mice.” Am J Pathol 154(3): 735-43.
Boeda, B., D. Weil, et al. (2001). “A specific promoter of the sensory cells of the inner ear defined by transgenesis.” Hum Mol Genet 10(15): 1581-9.
Bueler, H. (1999). “Adeno-associated viral vectors for gene transfer and gene therapy.” Biol Chem 380(6): 613-22.
Carter, P. J. and R. J. Samulski (2000). “Adeno-associated viral vectors as gene delivery vehicles.” Int J Mol Med 6(1): 17-27.
Carvalho, G. J. and A. K. Lalwani (1999). “The effect of cochleostomy and intracochlear infusion on auditory brain stem response threshold in the guinea pig.” Am J Otol 20(1): 87-90.
Derby, M. L., M. Sena-Esteves, et al. (1999). “Gene transfer into the mammalian inner ear using HSV-1 and vaccinia virus vectors.” Hear Res 134(1-2): 1-8.
During, M. J. and L. M. Ashenden (1998). “Towards gene therapy for the central nervous system.” Mol Med Today 4(11): 485-93.
Flotte, T., B. Carter, et al. (1996). “A phase I study of an adeno-associated virus-CFTR gene vector in adult CF patients with mild lung disease.” Hum Gene Ther 7(9): 1145-59.
Furness, D. N. and D. M. Lawton (2003). “Comparative distribution of glutamate transporters and receptors in relation to afferent innervation density in the mammalian cochlea.” J Neurosci 23(36): 11296-304.
Grimm, D., A. Kern, et al (1998). “Novel tools for production and purification of recombinant adenoassociated virus vectors.” Hum Gene Ther 9(18): 2745-60.
Grimm, D. and J. A. Kleinschmidt (1999). “Progress in adeno-associated virus type 2 vector production: promises and prospects for clinical use.” Hum Gene Ther 10(15): 2445-50.
Han, J. J., A. N. Mhatre, et al. (1999). “Transgene expression in the guinea pig cochlea mediated by a lentivirus-derived gene transfer vector.” Hum Gene Ther 10(11): 1867-73.
Ishimoto, S., K. Kawamoto, et al. (2002). “Gene transfer into supporting cells of the organ of Corti.” Hear Res 173(1-2): 187-97.
Jero, J., A. N. Mhatre, et al. (2001). “Cochlear gene delivery through an intact round window membrane in mouse.” Hum Gene Ther 12(5): 539-48.
Kawamoto, K., S. Ishimoto, et al. (2003). “Math1 gene transfer generates new cochlear hair cells in mature guinea pigs in vivo.” J Neurosci 23(11): 4395-400.
Kho, S. T., R. M. Pettis, et al. (2000). “Safety of adeno-associated virus as cochlear gene transfer vector: analysis of distant spread beyond injected cochleae.” Mol Ther 2(4): 368-73.
Klein, R. L., et al. (2002). “Measurements of vector-derived neurotrophic factor and green fluorescent protein levels in the brain.” Methods 28: 286-292.
Komeda, M., B. J. Roessler, et al. (1999). “The influence of interleukin-1 receptor antagonist transgene on spiral ganglion neurons.” Hear Res 131(1-2): 1-10.
Lalwani, A. K., B. J. Walsh, et al. (1996). “Development of in vivo gene therapy for hearing disorders: introduction of adeno-associated virus into the cochlea of the guinea pig.” Gene Ther 3(7): 588-92.
Lalwani, A. K., J. J. Han, et al. (1997). “Green fluorescent protein as a reporter for gene transfer studies in the cochlea.” Hear Res 114(1-2): 139-47.
Lalwani, A., B. Walsh, et al. (1998a). “Long-term in vivo cochlear transgene expression mediated by recombinant adeno-associated virus.” Gene Ther 5(2): 277-81.
Lalwani, A. K., B. J. Walsh, et al. (1998b). “Expression of adeno-associated virus integrated transgene within the mammalian vestibular organs.” Am J Otol 19(3): 390-5.
Loeb, J. E., W. S. Cordier, et al. (1999). “Enhanced expression of transgenes from adeno-associated virus vectors with the woodchuck hepatitis virus post-transcriptional regulatory element: implications for gene therapy.” Hum Gene Ther 10(14): 2295-305.
Luebke, A. E., J. D. Steiger, et al. (2001 a). “A modified adenovirus can transfect cochlear hair cells in vivo without compromising cochlear function.” Gene Ther 8(10): 789-94.
Luebke, A. E., P. K. Foster, et al. (2001b). “Cochlear function and transgene expression in the guinea pig cochlea, using adenovirus- and adeno-associated virus-directed gene transfer.” Hum Gene Ther 12(7): 773-81.
Mueller, K. L., Jacques, B. E., and Kelley, M. W. (2002). Fibroblast growth factor signaling regulates pillar cell development in the organ of Corti. J. Neurosci. 22: 9368-9377.
Muzyczka, N. (1992) Current Topics in Microbiol. and Immunol. 158:97-129.
Peel, A. L. and R. L. Klein (2000). “Adeno-associated virus vectors: activity and applications in the CNS.” J Neurosci Methods 98(2): 95-104.
Rabinowitz, J. E. and R. J. Samulski (2000). “Building a better vector: the manipulation of AAV virions.” Virology 278(2): 301-8.
Raphael, Y., J. C. Frisancho, et al. (1996). “Adenoviral-mediated gene transfer into guinea pig cochlear cells in vivo.” Neurosci Lett 207(2): 137-41.
Raphael, Y., K. N. Kobayashi, et al. (2001). “Severe vestibular and auditory impairment in three alleles of Ames waltzer (av) mice.” Hear Res 151(1-2): 237-249.
Raz, Y., and Kelley, M. W. (1999.). “Retinoic acid signaling is necessary for the development of the organ of Corti.” Dev. Biol. 213:180-193.
Rio, C., P. Dikkes, et al. (2002). “Glial fibrillary acidic protein expression and promoter activity in the inner ear of developing and adult mice.” J Comp Neurol 442(2): 156-62.
Snyder, R. O. (1999). “Adeno-associated virus-mediated gene delivery.” J Gene Med 1(3): 166-75.
Walters, R. W., S. M. Yi, et al. (2001). “Binding of adeno-associated virus type 5 to 2,3-linked sialic acid is required for gene transfer.” J Biol Chem 276(23): 20610-6.
Waring, M. D., C. W. Ponton, et al. (1999). “Activating separate ascending auditory pathways produces different human thalamic/cortical responses.” Hear Res 130(1-2): 219-29.
Xiao, X., J. Li, et al. (1997). “Gene transfer by adeno-associated virus vectors into the central nervous system.” Exp Neurol 144(1): 113-24.
Xiao, W., N. Chirmule, et al. (1999). “Gene therapy vectors based on adeno-associated virus type 1.” J Virol 73(5): 3994-4003.
Xu, L., et al. (2001). “CMV-beta-actin promoter directs higher expression from an adeno-associated viral vector in the liver than the cytomegalovirus or elongation factor 1 alpha promoter and results in therapeutic levels of human factor X in mice.” Hum. Gene Ther. 12: 563-573.
Yagi, M., E. Magal, et al. (1999). “Hair cell protection from aminoglycoside ototoxicity by adenovirus-mediated overexpression of glial cell line-derived neurotrophic factor.” Hum Gene Ther 10(5): 813-23.
Yamasoba, T., M. Yagi, et al. (1999). “Inner ear transgene expression after adenoviral vector inoculation in the endolymphatic sac.” Hum Gene Ther 10(5): 769-74.
Zheng, J. L. and W. Q. Gao (2000). “Overexpression of Math1 induces robust production of extra hair cells in postnatal rat inner ears.” Nat Neurosci 3(6): 580-6.
Zhu, H. Z., J. Q. Chen, et al. (2003). “Generation and characterization of transgenic mice expressing tamoxifen-inducible cre-fusion protein specifically in mouse liver.” World J Gastroenterol 9(8): 1844-7.
Zolotukhin, S., B. J. Byrne, et al. (1999). “Recombinant adeno-associated virus purification using novel methods improves infectious titer and yield.” Gene Ther 6(6): 973-85.
Zolotukhin, S., et al. (2002). “Production and purification of serotype 1, 2, and 5 recombinant adeno-associated viral vectors.” Methods 28: 158-167.
Zuo, J. (2002). “Transgenic and gene targeting studies of hair cell function in mouse inner ear.” J Neurobiol 53(2): 286-305.

1. A method of transducing a mammalian cochlear hair cell or support cell comprising:
delivering an adeno-associated virus (AAV) to the hair cell or support cell, wherein the AAV comprises DNA that is exogenous to the AAV and a promoter operatively linked to the DNA.

2. The method of claim 1, wherein the AAV is a high-titer virus of at least 10⁹ gp/μl.

3. The method of claim 1, wherein the DNA encodes a protein that promotes cochlear hair cell growth, or cell differentiation, or corrects a genetic mutation.

4. The method of claim 3, wherein the DNA encodes a Math1, Hath1, SOX2, connexin 26, or growth factor protein.

5. The method of claim 4, wherein the Math1, Hath1, or SOX2 protein is an ER receptor fusion protein.

6. The method of claim 1, wherein the cell is a support cell.

7. The method of claim 6, wherein the support cell is a Dieter's cell, Hensen's cell, pillar cell, inner phalangeal cell, border cell, or interdential cell.

8. The method of claim 7, wherein the promoter is a support cell specific promoter.

9. The method of claim 8, wherein the support cell specific promoter is a glial fibrillary acidic protein (GFAP) promoter, a excitatory amino acid transporter-1 (EAAT1) promoter, a glutamate transporter (GLAST) promoter or a murine cytomegalovirus (mCMV) promoter.

10. The method of claim 1, wherein the cochlear cell is a hair cell.

11. The method of claim 1, wherein the hair cell is an inner hair cell.

12. The method of claim 1, wherein the hair cell is an outer hair cell.

13. The method of claim 10, wherein the promoter is a hair cell specific promoter.

14. The method of claim 1, wherein the hair cell specific promoter is a human cytomegalovirus (CMV) promoter, a chicken β-actin/CMV hybrid (CAG) promoter, or myosin VIIA promoter.

15. The method of claim 14, wherein the promoter is the CAG promoter.

16. The method of claim 1, wherein both hair cells and support cells are transduced.

17. The method of claim 1, wherein the transduction efficiency is at least 30%.

18. The method of claim 1, wherein the AAV comprises serotype 1, 2, 6, or a mixture of two or more serotypes.

19. The method of claim 18, wherein the AAV comprises serotype 1.

20. The method of claim 18, wherein the AAV comprises serotype 2.

21. The method of claim 18, wherein the AAV comprises a mixture of AAV serotypes 1 and 2.

22. The method of claim 1, wherein the mammalian cell is a human cell.

23. The method of claim 22, wherein the human cell is in a living mammal.

24. The method of claim 1, wherein the AAV further comprises a woodchuck hepatitis virus post-transcription regulatory element (WPRE).

25. The method of claim 1, wherein the AAV is delivered by direct injection of the AAV into the cochlea.

26. A composition for transducing a mammalian cochlear hair cell or support cell comprising:
an adeno-associated virus (AAV) in an amount sufficient to transduce the hair cell or support cell, wherein the AAV comprises DNA that is exogenous to the AAV and that is operatively linked to a cochlear hair cell promoter or a support cell promoter.

27. The composition of claim 26, wherein the AAV is a high-titer virus of at least 10⁹ gp/μl.

28. The composition of claim 26, wherein the DNA encodes a Math1, Hath1, SOX2, connexin 26, or growth factor protein.

29. The composition of claim 28, wherein the Math1, Hath1, or SOX2 protein is an ER receptor fusion protein.

30. The composition of claim 26, wherein the support cell specific promoter is a glial fibrillary acidic protein (GFAP) promoter, a excitatory amino acid transporter-1 (EAAT1) promoter, a glutamate transporter (GLAST) promoter, or a murine cytomegalovirus (mCMV) promoter.

31. The composition of claim 30, wherein the hair cell specific promoter is a human cytomegalovirus (CMV) promoter, a chicken β-actin/CMV hybrid (CAG) promoter, or myosin VIIA promoter.

32. The composition of claim 31, wherein the promoter is the CAG promoter.

33. The composition of claim 26, wherein the AAV comprises serotype 1, 2, 6, or a mixture of two or more serotypes.

34. The composition of claim 33, wherein the AAV comprises a mixture of AAV serotypes 1 and 2.

35. The composition of claim 26, wherein the AAV further comprises a woodchuck hepatitis virus post-transcription regulatory element (WPRE).
